(19) United States
(12) Patent Application Publication
(10) Pub. No.: US 2026/0069120 A1
(43) Pub. Date: Mar. 12, 2026

(54) IMAGE CAPTURE AND ANALYSIS OF ANATOMIES

(71) Applicant: Welch Allyn, Inc., Skaneateles Falls, NY (US)

(72) Inventors: David G. Perkins, Tully, NY (US); Joshua Grant, Camillus, NY (US); Lei Guo, Manlius, NY (US); Allen R. Hart, Maryville, TN (US); Joshua Hess, Weedsport, NY (US); Matthew D. Mullin, Onondaga, NY (US); David Villarreal, Fayetteville, NY (US)

(21) Appl. No.: 19/322,839

(22) Filed: Sep. 9, 2025

Related U.S. Application Data
(60) Provisional application No. 63/692,841, filed on Sep. 10, 2024.

(51) Int. Cl. A61B 1/04; A61B 1/00; A61B 1/227

(52) U.S. Cl. CPC A61B 1/042; A61B 1/000096; A61B 1/00027; A61B 1/00048; A61B 1/00105; A61B 1/227; A61B 2560/0456

(57) ABSTRACT

A system for image capture and analysis of an anatomy includes an imaging device having a bracket for removably attaching the imaging device to an optical viewing device, a camera for capturing images through an eyepiece of the optical viewing device, a first display screen for displaying the images, a first transceiver for transferring the images, and a power source. The system includes a docking station having a docking port that supports the imaging device, a charger positioned inside the docking port for charging the power source of the imaging device, a second transceiver that receives the images, and a second display screen that displays the images. The second display screen of the docking station is larger than the first display screen of the imaging device.

### CROSS-REFERENCE TO RELATED APPLICATION(S)

This application claims the benefit of and priority to U.S. Provisional Patent Application No. 63/692,841, filed Sep. 10, 2024, the entire disclosure of which is incorporated by reference herein in its entirety.

### BACKGROUND

Optical viewing devices such as otoscopes for viewing the ears of a patient, ophthalmoscopes for viewing the eyes of a patient, and dermatoscopes for viewing the skin of a patient are typically used during routine physical examinations of patients. When a physician is using an optical viewing device to view an anatomy of a patient, it can be desirable for the patient and/or a companion of the patient to view images and video streams of the anatomy. Also, it can be desirable to share the images and video streams of the anatomy viewed from an optical viewing device for teaching students, assistants, and apprentices.

### SUMMARY

In general terms, the present disclosure relates to image capture and analysis of anatomies. In one possible configuration, a docking station receives images captured by an imaging device attached to an optical viewing device, and the docking station displays and analyzes the images. Various aspects are described in this disclosure, which include, but are not limited to, the following aspects.

One aspect relates to a system for image capture and analysis of an anatomy, the system comprising: an imaging device including: a bracket that removably attaches the imaging device to an optical viewing device; a camera that captures images through an eyepiece of the optical viewing device; a first display screen that displays the images captured by the camera; a first transceiver that transfers the images captured by the camera to another device; and a power source that powers the camera, the first display screen, and the first receiver; and a docking station including: docking port that supports the imaging device; a charger positioned inside the docking port for charging the power source of the imaging device; a second transceiver that receives the images captured by the camera of the imaging device; a second display screen that displays the images captured by the camera of the imaging device, the second display screen of the docking station being larger than the first display screen of the imaging device; and a controller including a processing circuitry having a memory storing instructions which, when executed by the processing circuitry, cause the processing circuitry to: receive the images captured by the camera of the imaging device; and display the images on the second display screen.

Another aspect relates to a method of image capture and analysis of an anatomy, the method comprising: communicating data identifying a patient to an imaging device, the imaging device removably attached to an optical viewing device, the optical viewing device includes at least one of an ophthalmoscope, an otoscope, and a dermatoscope; receiving images of the anatomy from the imaging device, the images being captured by a camera of the imaging device that captures the images viewed through an eyepiece of the optical viewing device; displaying the images of the anatomy on a display screen on a docking station; and analyzing the images of the anatomy.

Another aspect relates to a docking station for an imaging device that is configured to capture images through an eyepiece of an optical viewing device, the docking station comprising: docking port configured to support the imaging device; a charging unit positioned inside the docking port for charging a power source of the imaging device; a transceiver for receiving images captured by a camera of the imaging device; a display screen configured to display the images received from the imaging device; and a controller including a processing circuitry having a memory storing instructions which, when executed by the processing circuitry, cause the processing circuitry to: receive data identifying the patient; communicate the data identifying the patient to the imaging device; receive the images captured by the camera of the imaging device, the images being received as raw, unprocessed data; display the images on the display screen; and analyze the images captured by the camera of the imaging device.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a rear isometric view of an example of an imaging device attached to an optical viewing device.

FIG. 2 is a front isometric view of the imaging device before attachment to the optical viewing device of FIG. 1.

FIG. 3 schematically illustrates an example of the optical viewing device of FIG. 1.

FIG. 4 schematically illustrates an example of a docking station connected to the imaging device of FIGS. 1 and 2.

FIG. 5 illustrates an example of a system that includes the imaging device of FIGS. 1 and 2 and a first example of the docking station of FIG. 4.

FIG. 6 illustrates another example of a system that includes the imaging device of FIGS. 1 and 2 and a second example of the docking station of FIG. 4.

FIG. 7 illustrates another example of a system that includes the imaging device of FIGS. 1 and 2 and a third example of the docking station of FIG. 4.

FIG. 8 illustrates another example of a system that includes the imaging device of FIGS. 1 and 2 and a fourth example of the docking station of FIG. 4.

FIG. 9 schematically illustrates an example of a method of image capture and analysis of an anatomy that can be performed by any of the systems of FIGS. 5-8.

### DETAILED DESCRIPTION

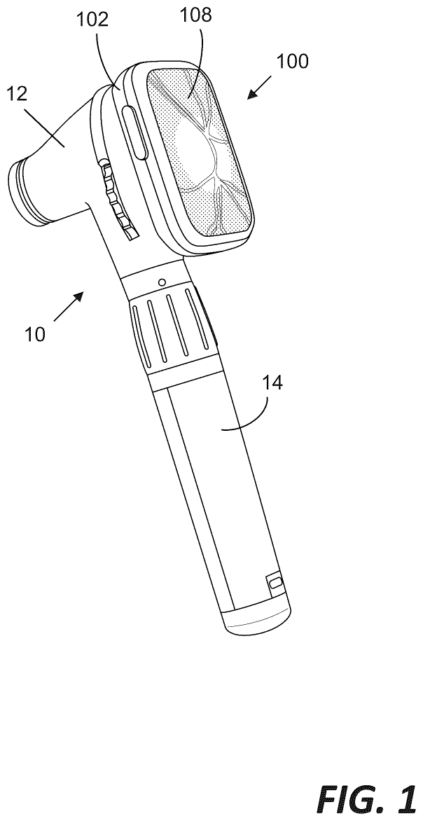

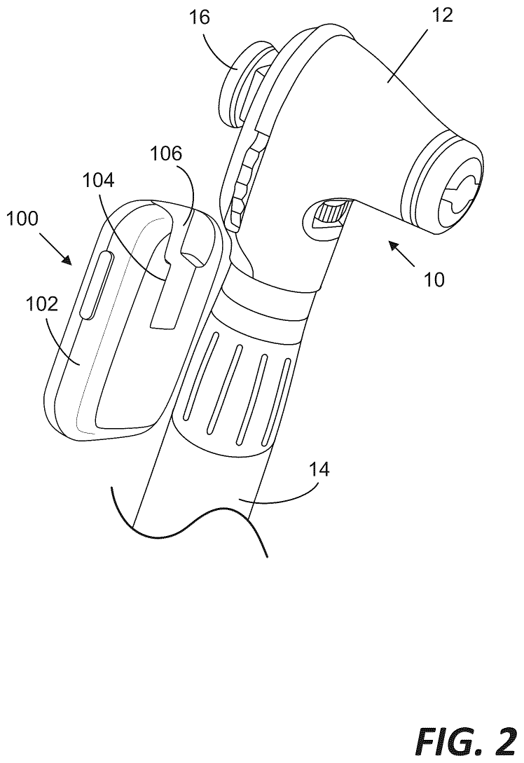

FIG. 1 is a rear isometric view of an example of an imaging device 100 attached to an optical viewing device 10. FIG. 2 is a front isometric view of the imaging device 100 before attachment to the optical viewing device 10. The imaging device 100 is configured to removably attach to the optical viewing device 10 such that the imaging device 100 can attach and detach from the optical viewing device 10 as desired. The optical viewing device 10 is configured for viewing an anatomy of a patient such as the eyes, ears, and skin surfaces of the patient.

In the example provided in FIGS. 1 and 2, the optical viewing device 10 is an ophthalmoscope that is used by a physician to see inside the fundus of an eye and to view other structures of the eye. The physician uses the ophthalmoscope during an eye examination for determining the health of the retina, the optic disc, and other structures of the eyes that may be performed as part of a routine physical examination of a patient. The imaging device 100 can also attach to other types of optical viewing devices such as otoscopes for viewing inside the ears of the patient, dermatoscopes for viewing skin surfaces of the patient, and the like.

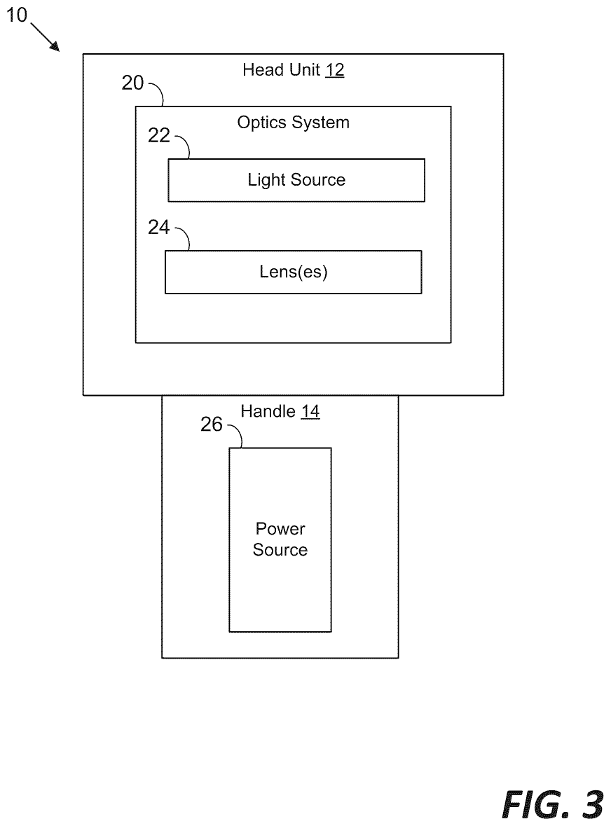

FIG. 3 schematically illustrates an example of the optical viewing device 10. Referring now to FIGS. 1-3, the optical viewing device 10 includes a head unit 12 removably attached to a handle 14. The head unit 12 can include an optics system 20 that includes a light source 22 for directing light toward an anatomy. As an illustrative example, the light source 22 can include one or more light-emitting diodes (LEDs). The optics system 20 further includes one or more lenses 24 such as magnifying lenses, filters, and the like. The optical viewing device 10 includes an eyepiece 16 (see FIG. 2) connected to the optics system 20 for viewing an anatomy.

The handle 14 removably attaches to the head unit 12. The handle 14 includes a power source 26 that powers one or more components of the optics system 20 on the head unit 12. The handle 14 can include rechargeable batteries, disposable batteries, or a tether to a wall transformer for supplying electrical power to the head unit 12.

The imaging device 100 is a portable, battery powered device that captures high quality images and video streams from the optical viewing device 10. As shown in FIGS. 1 and 2, the imaging device 100 includes a housing 102 having a bracket 104 that removably attaches to the eyepiece 16 of the head unit 12. The imaging device 100 includes a camera 106 that captures images viewed through the eyepiece 16 of the head unit 12. The camera 106 of the imaging device 100 is configured for alignment with the eyepiece 16 of the head unit 12 for capturing the images and video streams viewed through the eyepiece 16 of the head unit 12.

The imaging device 100 includes a display screen 108 to display the images and video streams captured by the camera 106. The display screen 108 provides an area for viewing the images and video streams of anatomical structures that is larger than that of the eyepiece 16 of the head unit 12. Further, the display screen 108 allows the physician to view the anatomical structures without having to position their face against the eyepiece 16 which improves their freedom of movement during physical examination of the patient.

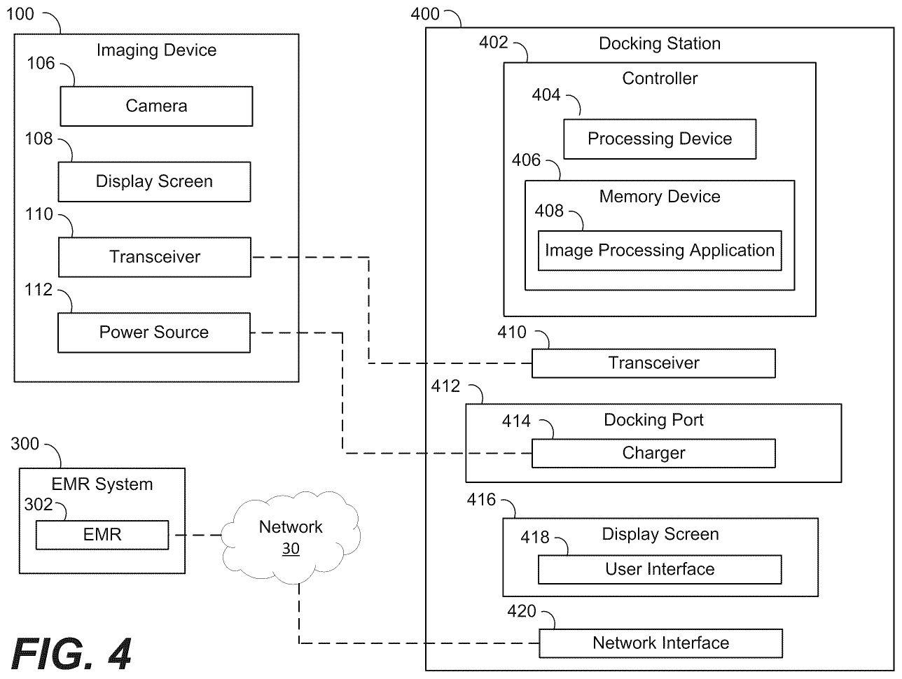

FIG. 4 schematically illustrates an example of a docking station 400 connected to the imaging device 100. The docking station 400 includes a controller 402 having a processing device 404 and a memory device 406. In some examples, the processing device 404 and the memory device 406 can be part of a processing circuitry that executes instructions causing the processing circuitry to perform the functions of the docking station 400 described herein.

The processing device 404 is an example of a processing unit such as a central processing unit (CPU). The processing device 404 can include one or more central processing units (CPUs). In some examples, the processing device 404 can include one or more digital signal processors, field-programmable gate arrays, or other electronic processing devices.

The memory device 406 operates to store data and instructions for execution by the processing device 404. The memory device 406 includes computer-readable media, which may include any media that can be accessed by the processing device 404. By way of illustrative example, the computer-readable media can include computer-readable storage media and computer-readable communication media. As shown in FIG. 4, the memory device 406 stores an image processing application 408, which will be described in more detail below. In some examples, the memory device 406 can store the images and video streams captured by the camera 106 of the imaging device 100 locally on the docking station 400.

The computer-readable storage media includes volatile and nonvolatile, removable and non-removable media implemented in any device configured to store information such as computer-readable instructions, data structures, program modules, or other data. The computer-readable storage media can include, but is not limited to, random access memory, read only memory, electrically erasable programmable read only memory, flash memory, and other memory technology, including any medium that can be used to store information that can be accessed by the processing device 404. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are within the scope of computer-readable media.

The docking station 400 includes a transceiver 410 that communicates with a transceiver 110 on the imaging device 100 for receiving data from the imaging device 100 such as the images and video streams captured by the camera 106 of the imaging device 100. Also, the transceiver 410 can transmit data to the transceiver 110 on the imaging device 100 such as data identifying a patient who is undergoing an examination performed by use of the optical viewing device 10 and the imaging device 100 attached thereto. In further examples, the transceiver 410 can transmit adjustments of one or more settings, software updates, and the like to the transceiver 110 on the imaging device 100. Accordingly, the transceiver 110 on the imaging device 100 and the transceiver 410 on the docking station 400 enable bidirectional communications between the imaging device 100 and the docking station 400. In some examples, the transceivers 110, 410 wirelessly transmit and receive data through a wireless connection such as via Wi-Fi, ultra-wideband (UWB), Bluetooth, and other types of wireless communication protocols and technologies. In such examples, the transceivers 110, 410 are wireless antennas.

The transceiver 110 of the imaging device 100 can pair to a transceiver 410 of a docking station 400 that is in closest proximity to the imaging device 100 in environments where there are a plurality of imaging devices 100 and a plurality of docking stations 400. In some examples, a handshake is performed to authenticate the imaging device 100 and the docking station 400 before the devices are paired together, and to coordinate the transmissions between the transceiver 110 of the imaging device 100 and the transceiver 410 of the docking station 400.

In some examples, the display screen 108 on the imaging device 100 and the display screen 416 on the docking station 400 are altered to show that they have been paired together.

For example, the display screens 108, 416 can be altered to have the same color background (e.g., blue) to indicate that the imaging device 100 and the docking station 400 are paired together. When the display screens 108, 416 have different color backgrounds (e.g., green versus yellow), this indicates that the imaging device 100 and the docking station 400 are not paired together. Alternatively, the display screens 108, 416 can flash or blink for a predetermined period of time to show that they have been successfully paired together.

Alternatively, the display screens 108, 416 can display a matching label such as a room number, physician name, or patient name to show that the imaging device 100 and the docking station 400 have been correctly paired together. In such examples, when the display screens 108, 416 display different room numbers, physician names, or patient names, this indicates that the imaging device 100 and the docking station 400 are not paired together.

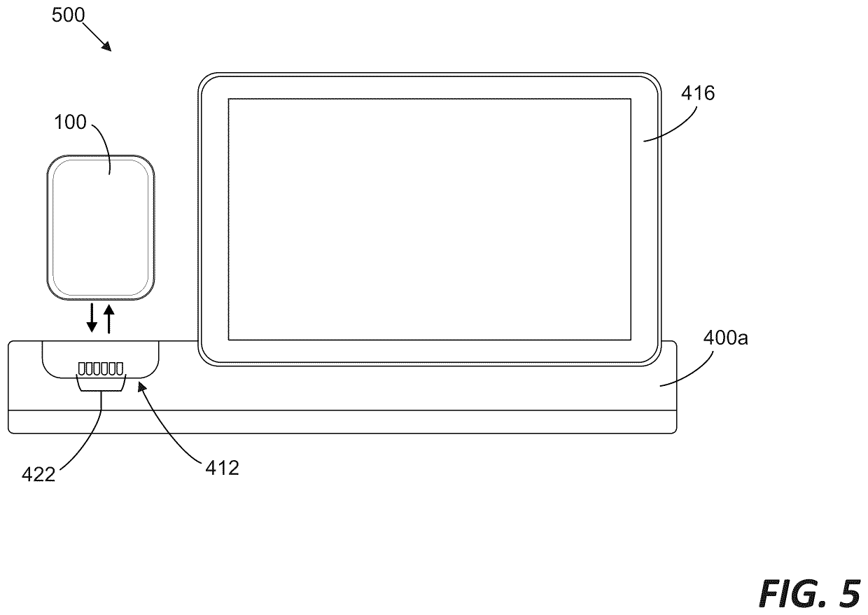

The docking station 400 includes a docking port 412 that receives the imaging device 100 such as for stowage of the imaging device 100 when it is not being used. As shown in FIGS. 5-8, which will be described in more detail further below, the docking port 412 can support the imaging device 100 in an upright position for stowage when not being used.

As further shown in FIG. 4, a charger 414 is positioned inside the docking port 412 for charging a power source 112 on the imaging device 100. The power source 112 supplies electrical power to the camera 106, the display screen 108, the transceiver 110, and other components on the imaging device 100. The power source 112 can include one or more rechargeable batteries that are charged by the charger 414 of the docking station 400 when the imaging device 100 is stowed inside the docking port 412 of the docking station 400.

In further examples, the docking station 400 can include one or more additional docking ports and chargers such as one or more docking ports for receiving one or more handles 14 of one or more of the optical viewing devices 10. In such examples, the one or more additional docking ports can each include a charger for charging the power source 26 in the handle 14 of the optical viewing device 10 when stowed inside the docking port.

The docking port 412 can include a set of electrical contacts 422 (see FIGS. 5-8) that are configured to engage a corresponding set of electrical contacts on the imaging device 100. For example, when imaging device 100 is placed in the docking port 412, the set of electrical contacts 422 establishes an electrical connection with the corresponding set of electrical contacts on the imaging device 100 for recharging the power source 112 on the imaging device 100.

In some examples, the electrical connection established between the set of electrical contacts 422 in the docking port 412 and the corresponding set of electrical contacts on the imaging device 100 provides a wired data communication pathway as alternative means for transmitting the images and video streams from the imaging device 100 to the docking station 400. The wired data communication pathway can increase a transmission speed for transmission of the images and video streams from the imaging device 100 to the docking station 400.

Alternatively, the docking port 412 can wirelessly recharge the power source 112 of the imaging device 100 such as by using magnetic induction that uses an electromagnetic field to transfer energy between the docking station 400 and the imaging device 100. In such examples, the docking port 412 does not need to include the set of electrical contacts 422 to establish an electrical connection with the corresponding set of electrical contacts on the imaging device 100. Accordingly, the docking port 412 can include wired interfaces and/or wireless interfaces for recharging the power source 112 on the imaging device 100.

The docking station 400 includes a display screen 416 that displays a user interface 418. The display screen 416 can include a touchscreen such that the user interface 418 operates to receive inputs from a physician. The display screen 416 on the docking station 400 is larger than the display screen 108 on the imaging device 100. The larger size of the display screen 416 on the docking station 400 provides easier viewing of data from an electronic medical record (EMR) 302 (see FIG. 4) than on the display screen 108 on the imaging device 100.

In some examples, the display screen 416 receives annotations before, during, or after examining a patient by use of the optical viewing device 10 and the imaging device 100 attached thereto. The annotations can be drawn over the images and video streams captured by the camera 106 of the imaging device 100 to identify one or more areas interest, clinical observations, or potential diagnoses. For example, the annotations can be drawn or written over the images and video streams by the physician using their fingers or a stylus. The annotations can further include clinical observations, potential diagnoses, and notes that are typed or otherwise entered into the user interface 418 displayed on the display screen 416 of the docking station 400.

The images and video streams along with the annotations can be stored locally on the memory device 406 of the docking station 400. Additionally, or alternatively, the annotations, the images, and the video streams can be stored in an external database such as in a cloud-based storage, or in an electronic medical record (EMR) maintained by an EMR system (see FIG. 4).

The user interface 418 can further display the images and video streams captured by the camera 106 of the imaging device 100 during a physical examination of a patient. In such instances, the user interface 418 allows the patient or one or more companions of the patient (e.g., family members, relatives, friends, and the like) to view the images and video streams captured by the camera 106 of the imaging device 100 while the imaging device 100 is being used by the physician during a physical examination of the patient. The one or more images and video streams displayed on the display screen 416 can show or explain a diagnosis or clinical observation made by the physician during the physical examination for better understanding.

As further shown in FIG. 4, the docking station 400 includes a network interface 420 that allows the docking station 400 to connect to one or more networks such as a network 30. The network interface 420 allows the docking station 400 to communicate over the network 30 with external systems and devices. For example, the network interface 420 allows the docking station 400 to communicate over the network 30 with an electronic medical record (EMR) system 300. The docking station 400 can transmit the images, the video streams, the annotations, and the analyses of the images and/or video streams, and other data over the network 30 for storage in an external database such as in a cloud-based storage, or in the EMR of the patient maintained on the EMR system. The docking station 400 can transmit the images, the video streams, the annotations, and the analyses of the images and/or video streams, and the other data using Substitutable Medical Applications and Reusable Technologies (SMART) on Fast Healthcare Interoperability Resources (FIHR), or other health information transfer protocols.

FIG. 5 illustrates an example of a system 500 for viewing an anatomy of a patient. The system 500 includes the imaging device 100 and a first example of the docking station 400a. The system 500 can further include the optical viewing device 10. In this example, the display screen 416 is integrated with the docking station 400a such that they form a single device.

As shown in FIG. 5, the imaging device 100 can be placed in the docking port 412 for stowage such as in an upright position. Further, the docking port 412 includes the set of electrical contacts 422 for recharging the power source 112 on the imaging device 100. In some examples, the set of electrical contacts 422 provide a wired data communication pathway for transmitting the images and video streams from the imaging device 100 to the docking station 400a, and/or transmitting data and/or instructions to the docking station 400a to the imaging device 100. In the example of FIG. 5, a wireless connection can be established between the imaging device 100 and the docking station 400a such as to transmit images and video streams captured by the camera 106 of the imaging device 100 while the imaging device is being used during an examination.

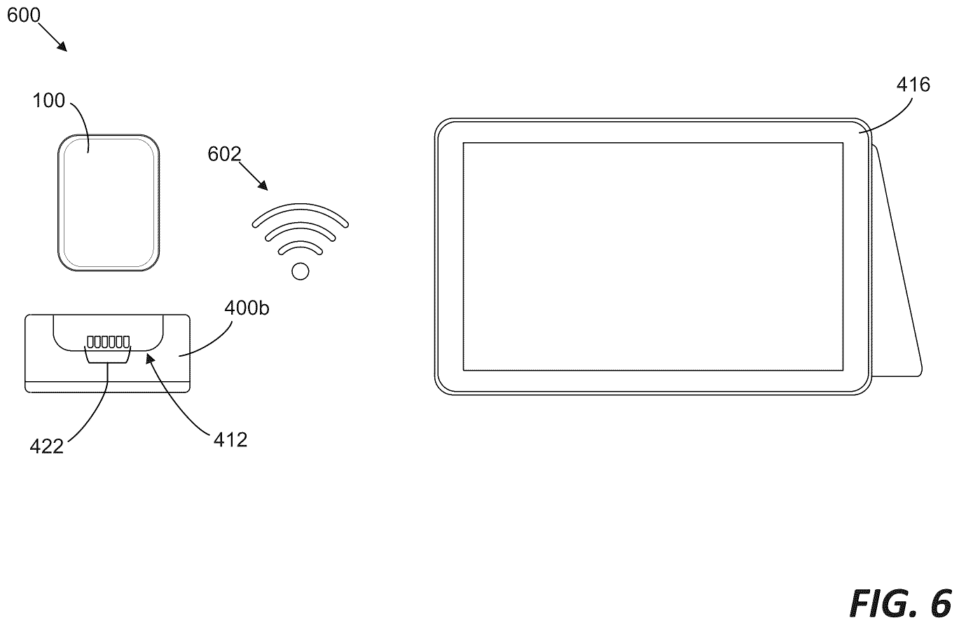

FIG. 6 illustrates another example of a system 600 for viewing an anatomy of a patient. The system 600 includes the imaging device 100 and a second example of the docking station 400b. The system 600 can further include the optical viewing device 10. Like in the example described above, the docking station 400b includes the docking port 412 for stowing the imaging device 100 in an upright position, and the set of electrical contacts 422 for recharging the power source 112 on the imaging device 100. However, in this example, the display screen 416 is physically separate from the docking station 400b. For example, the display screen 416 can belong to a separate device independent of the docking station 400b such as a workstation computer, a tablet computer, a laptop or notebook computer, a smartphone, and the like.

In the example of FIG. 6, the imaging device 100, the docking station 400b, and the display screen 416 communicate with one another via wireless connections 602 such as Wi-Fi, Bluetooth, or another type of wireless communication protocol. The wireless connections 602 can be established by one or more transceivers such as the transceiver 110 on the imaging device 100 and the transceiver 410 on the docking station 400, and/or other additional transceivers.

The imaging device 100 can transmit the images and the video streams captured by the camera 106 to the docking station 400b via a wireless connection 602, and the docking station 400b transmits the images and the video streams to the display screen 416 via another wireless connection 602. Alternatively, the imaging device 100 can transmit the images and the video streams directly to the display screen 416 via a wireless connection 602.

The display screen 416 can transmit data and/or instructions to the docking station 400b via a wireless connection 602, and the docking station 400b can transmit the data and/or instructions to the imaging device 100 via another wireless connection 602. Alternatively, the display screen 416 can transmit the data and/or instructions directly to the imaging device 100 via a wireless connection 602. An example of the data that can be transmitted from the docking station 400 and/or the display screen 416 to the imaging device 100 includes an identity of a patient who is undergoing examination by use of the optical viewing device 10 attached to the imaging device 100 such that the identity of the patient can be displayed on the display screen 108 of the imaging device 100 while being used during an examination of the patient.

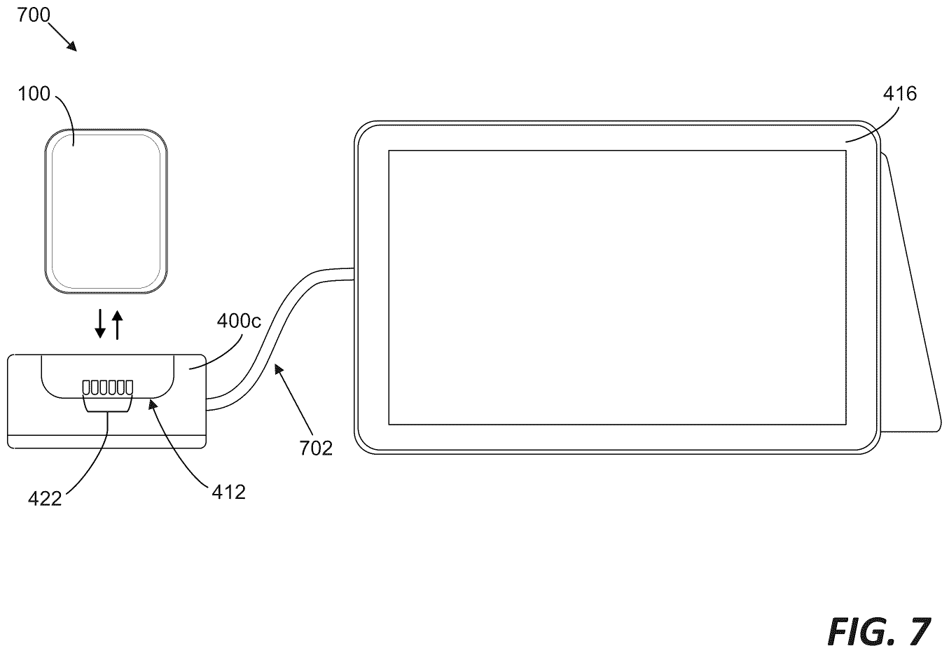

FIG. 7 illustrates another example of a system 700 for viewing an anatomy of a patient. The system 700 includes the imaging device 100 and a third example of the docking station 400c. The system 700 can further include the optical viewing device 10. Like in the examples described above, the docking station 400c includes the docking port 412 for stowing the imaging device 100 in an upright position, and the set of electrical contacts 422 for recharging the power source 112 on the imaging device 100 when stowed in the docking port.

In the example illustrated in FIG. 7, the display screen 416 belongs to a device that is independent of the docking station 400c, but that is physically connected to the docking station 400c via a wired connection 702. For example, the display screen 416 can belong to a separate device such as a workstation computer, a tablet computer, a laptop or notebook computer, a smartphone, and the like. The docking station 400c can plug into the separate device of the display screen 416. In some examples, the separate device of the display screen 416 transmits electrical power through the wired connection 702 for charging the power source 112 of the imaging device 100 via the set of electrical contacts 422 in the docking port 412 when the imaging device 100 is placed inside the docking port 412 for stowage.

In the example of FIG. 7, the imaging device 100 can communicate with the docking station 400c via a wireless connection established between the transceiver 110 on the imaging device 100 and the transceiver 410 on the docking station 400. For example, the imaging device 100 transmits the images and the video streams captured by the camera 106 to the docking station 400c via the wireless connection between the transceivers 110, 410, and the docking station 400c transmits the images and the video streams to the display screen 416 via the wired connection 702. Alternatively, the imaging device 100 can transmit the images and the video streams directly to the display screen 416 via a wireless connection between the transceiver 110 on the imaging device 100 and a transceiver on the display screen 416.

Additionally, the display screen 416 can transmit data and instructions to the docking station 400c via the wired connection 702, and the docking station 400c can transmit the data and instructions to the imaging device 100 via the wireless connection established between the transceiver 110 on the imaging device 100 and the transceiver 410 on the docking station 400.

Alternatively, the display screen 416 can transmit the data and instructions directly to the imaging device 100 via a wireless connection. The data can include an identity of a patient who is undergoing examination by use of the optical viewing device 10 attached to the imaging device 100 such that the identity of the patient can be displayed on the display screen 108 of the imaging device 100 while being used during an examination of the patient.

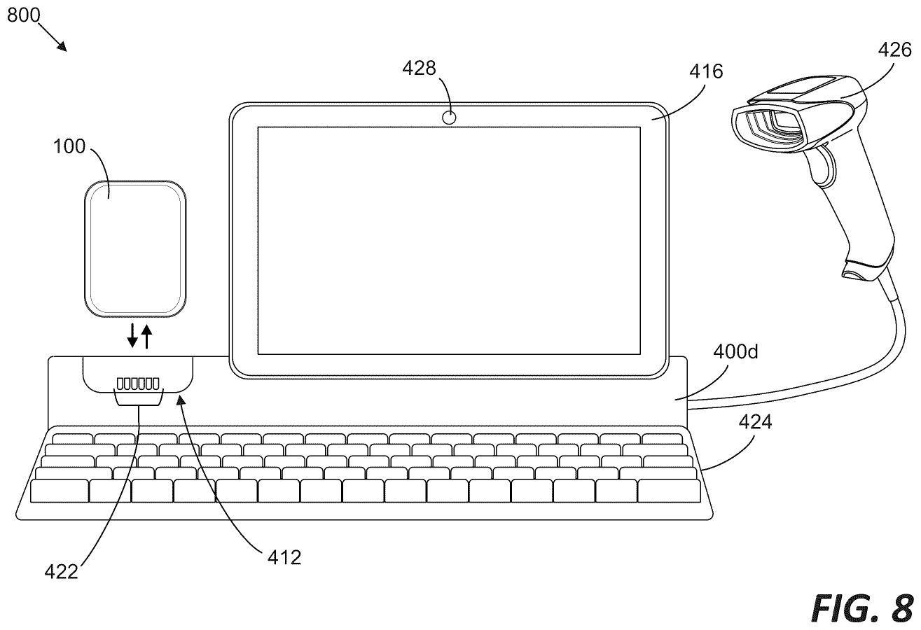

FIG. 8 illustrates another example of a system 800 for viewing an anatomy of a patient. The system 800 including the imaging device 100 and a fourth example of the docking station 400d. The system 800 can further include the optical viewing device 10.

The system 800 is similar to the system 500 of FIG. 5 in that the display screen 416 is integrated with the docking station 400d such that they form a single device. Further, in this example, the docking station 400d includes additional input devices such as a keyboard 424 that can be integrated with the docking station 400d or that can connect to the docking station 400d such as via a wired plug-in connection or a wireless connection. Alternatively, the keyboard 424 can be a virtual keyboard displayed on the display screen 416, which is a touchscreen. The keyboard 424 can be used to type annotations, clinical observations, and other notes.

The docking station 400d further includes a scanner 426. In the example shown in FIG. 8, the scanner 426 is connected to the docking station 400d by a wired plug-in connection. In alternative examples, the scanner 426 can connect to the docking station 400d via a wireless connection. The scanner 426 can be used to scan machine-readable labels such as quick-response (QR) codes, barcodes, and the like that encode information such as an identity of a patient who is undergoing an examination. Alternatively, or additionally, the docking station 400d can include a built-in camera 428 that can be used to scan the machine-readable labels to identify the patient. The machine-readable labels can be attached to a physical object associated with the patient such as folder or file, or to an object that is worn by the patient such as a wristband or a bracelet.

In yet further examples, the transceiver 410 on the docking station 400 can detect a radio frequency identification (RFID) tag attached to an object associated with the patient for automatically identifying the patient wirelessly. Once the identity of the patient is read or otherwise detected by the docking station 400, the transceiver 410 on the docking station 400 can communicate the identity of the patient to the transceiver 110 on the imaging device 100.

Additional types of input and out devices such as a mouse to move a cursor on the display screen 416, and a printer to print one or more images captured by the camera 106 of the imaging device 100 can be integrated with or otherwise connect to the docking station 400d.

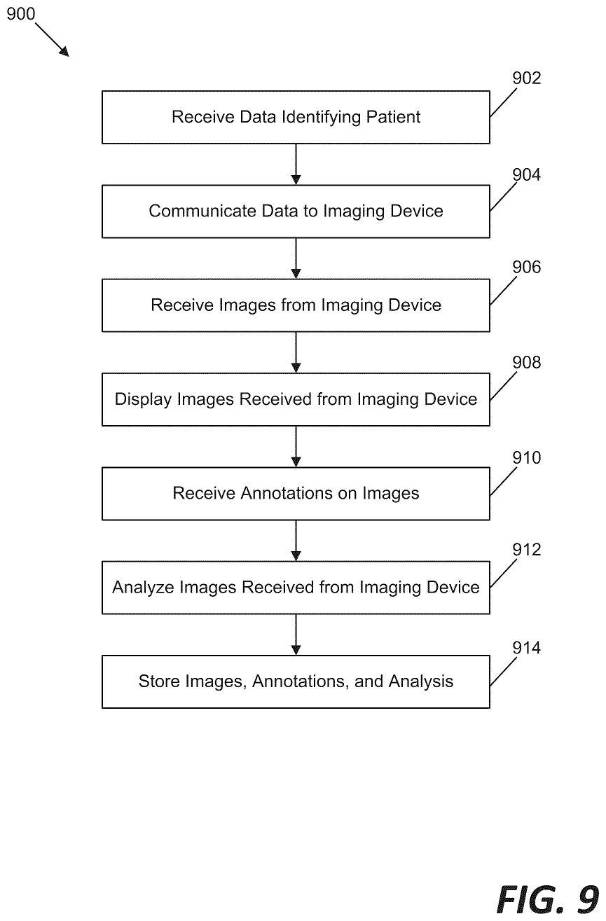

FIG. 9 schematically illustrates an example of a method 900 of image capture and analysis of an anatomy. The method 900 can be performed by any of the systems 500-800 of FIGS. 5-8. The method 900 includes an operation 902 of receiving data identifying a patient. The data identifying the patient can be received by the docking station 400 by one or more inputs received on the display screen 416 of the docking station 400. For example, the data identifying the patient can be received as a result of a user such as a physician selecting the patient from a list of patients displayed on the display screen 416. The list of patients can be generated as part of a schedule of appointments in an office or clinic where the docking station 400 is located.

Alternatively, or additionally, the data identifying the patient can be pulled directly from the EMR 302 of the patient maintained by the EMR system 300.

Alternatively, the data identifying the patient can be received by detection of machine-readable data. For example, operation 902 can include receiving the data identifying the patient from the scanner 426 or the built-in camera 428 reading a machine-readable label such as a QR code, a barcode, and the like. In further examples, operation 902 can include automatically detecting the identity of the patient via the transceiver 410 on the docking station 400 detecting an RFID tag attached to an object associated with the patient. In some examples, the camera 106 on the imaging device 100 can be used to read the machine-readable label to identify the patient.

The method 900 includes an operation 904 of communicating the data identifying the patient to the imaging device 100. Operation 904 can occur when the imaging device 100 is attached to an optical viewing device 10 such as an ophthalmoscope, an otoscope, or a dermatoscope. Operation 904 can include transmitting the data identifying the patient from the transceiver 410 on the docking station 400 to the transceiver 110 on the imaging device 100.

Alternatively, operation 904 can occur before the imaging device 100 is removably attached to the optical viewing device 10 such as when the imaging device 100 is stowed inside the docking port 412 of the docking station 400. In such examples, the data identifying the patient can be communicated through a wired connection such as from the set of electrical contacts 422 in the docking port 412 to the corresponding set of electrical contacts on the imaging device 100 when the imaging device is being stowed inside the docking port 412.

The method 900 includes an operation 906 of receiving one or more images and/or a video stream of the anatomy from the imaging device 100. The images and video stream of the anatomy are captured by the camera 106 of the imaging device 100. As described above, the camera 106 captures the images and video stream of the anatomy viewed through the eyepiece 16 of the optical viewing device 10 when the imaging device 100 is attached to the optical viewing device 10. In some examples, the one or more images and/or the video stream of the anatomy is received wirelessly such as from a transmission of the transceiver 110 on the imaging device 100 that is received by the transceiver 410 on the docking station 400. Alternatively, the one or more images and/or the video stream of the anatomy can be received through a wired connection such as from a transmission of the set of electrical contacts on the imaging device 100 that is received by the set of electrical contacts 422 in the docking port 412 when the imaging device 100 is returned to the docking port 412 for stowage.

The method 900 includes an operation 908 of displaying the one or more images and/or the video stream of the anatomy on the display screen 416 of the docking station 400. Operation 908 can also include simultaneously displaying the one or more images and/or the video stream of the anatomy on the display screen 108 of the imaging device 100. The display screen 416 on the docking station 400 is larger than the display screen 108 on the imaging device 100 allowing the patient and/or a companion of the patient to view the images and/or the video stream of the anatomy while the physician simultaneously views the images and/or the video stream on the display screen 108 of the imaging device 100 while examining the anatomy using the optical viewing device 10 (e.g., ophthalmoscope, otoscope, or dermatoscope).

The method 900 includes an operation 910 of receiving annotations on the user interface 418 displayed on the display screen 416 of the docking station 400. The display screen 416 includes a touchscreen such that a user such as a physician can use their fingers or a stylus to draw or write one or more annotations over the images or video stream such as to identify one or more areas interest, clinical observations, or potential diagnoses. Also, the annotations can be typed or otherwise entered on the user interface 418 by using the keyboard 424.

The annotations can be received during or after examining the anatomy of the patient by use of the optical viewing device 10 and the imaging device 100 attached thereto. In some examples, the annotations can be received prior to examining the anatomy of the patient such as when the annotations are drawn, typed, or entered on the user interface 418 displayed on the display screen 416 based on previously taken images or video streams that are accessible for viewing on the display screen 416 of the docking station 400 prior to examination. For example, a screen of the EMR 302 of the patient can be displayed on the display screen 416 of the docking station 400, and the EMR 302 view can include the previously taken images or video streams of the patient. The annotations can also be drawn, typed, or entered on the user interface 418 when interviewing the patient regarding their symptoms prior to using the optical viewing device 10.

The method 900 includes an operation 912 of executing the image processing application 408 for analysis of the one or more images and/or the video stream of the anatomy captured by the camera 106 of the imaging device 100. As discussed above, the image processing application 408 is stored on the memory device 406 of the docking station 400. In some examples, the imaging device 100 does not analyze the images and/or video streams captured by the camera 106. Instead, all of the analysis of the images and/or video streams captured by the camera 106 is performed on the docking station 400. In alternative examples, computationally intensive applications (i.e., applications that require a large amount of computing resources such as processing power, memory, and time to perform particular computational tasks or algorithms) are performed on the docking station 400, while computationally light applications are performed on the imaging device 100. In some further examples, the computationally intensive applications can be performed by another system located separately from the imaging device 100 and the docking station 400 such as by an external server such as a cloud-based server, or other type of external computing resource.

Functions performed on the imaging device 100 can include autofocusing, auto-sizing, and/or stabilizing the images captured by the camera 106 for display on the display screen 108. The imaging device 100 can be programmed to apply digital filters such as red-free or high contrast digital filters. Further, the imaging device 100 can perform basic image signal processing functions such as auto white balance (AWB), high dynamic range (HDR) correction, image sharpening, noise reduction, auto-exposure, and/or demosaicing (or de-mosaicing, demosaicking), also known as color reconstruction, which is a digital image processing algorithm used to reconstruct a full color image from the incomplete color samples output from an image sensor overlaid with a color filter array (CFA) such as a Bayer filter.

Additionally, the imaging device 100 can down-select or recommend images (or frames from a video stream) for transferring to the docking station 400 for further processing such as images and/or frames that are in focus and that provide a view of target anatomy. The images and/or frames that are not in focus or that are not of a target anatomy are discarded.

Advantageously, this reduces the amount of data transferred from the imaging device 100 to the docking station 400 which can improve transfer speeds especially when the imaging device 100 and the docking station 400 are wirelessly connected like in the example shown in FIG. 6.

The imaging device 100 can also be programmed to detect machine-readable labels (e.g., barcodes, QR codes, and the like) such as for recognizing an identification of a patient. The imaging device 100 can also be programmed to perform simple algorithms such as to detect a type of head unit that the imaging device 100 is attached to based on image properties such as to differentiate between attachment to an otoscope, an ophthalmoscope, and a dermatoscope.

By performing the image processing application 408 on the docking station 400 instead of on the imaging device 100, the computational requirements for the imaging device 100 are reduced such that a smaller, less sophisticated type of controller can be used on the imaging device 100 instead of the controller 402 of the docking station 400. The smaller, less sophisticated controller installed on the imaging device 100 allows for a reduction in size and weight of the imaging device 100 such that the imaging device 100 is not bulky or heavy when attached to the optical viewing device 10. The reduced size and weight of the imaging device 100 mitigates interference with the use of the optical viewing device 10 for viewing an anatomy when the imaging device 100 is attached to optical viewing device 10. Also, by performing the image processing application 408 on the docking station 400 instead of on the imaging device 100, the battery life of the power source 112 of the imaging device 100 can be improved.

The imaging device 100 provides a lightweight device for image capture and display for examining an anatomy of a patient, while the docking station 400 enables a user to enter data and other exam details before, during, or after an examination of an anatomy, and to analyze and/or annotate the images and/or the video streams of the anatomy. Further, data entry on the display screen 108 of the imaging device 100 (which is smaller than the display screen 416 of the docking station 400) is mitigated or eliminated, which improves the ergonomics of the system because instead of data entry on a smaller screen, the data entry is done on a larger screen.

In some examples, the image processing application 408 includes artificial intelligence for processing and/or analyzing the images and video streams captured by the camera 106 of the imaging device 100. Artificial intelligence such as machine learning models can be computationally intensive, and as such is performed on the docking station 400.

The image processing application 408 can analyze the images captured by the camera 106 of the imaging device 100 for screening for one or more disease states or conditions. For example, when the imaging device 100 is attached to an ophthalmoscope, the image processing application 408 can analyze the images captured by the imaging device 100 for screening of optic disc swelling such as papilledema, diabetic eye disease, retinopathy of prematurity, macular edema, retinal tears, retinal detachment, glaucoma, and other eye conditions.

In some further examples, the image processing application 408 can pull pre-existing images of a patient from the EMR 302 of the patient or from another storage location (e.g., cloud-based storage), and the image processing application 408 can compare the pre-existing images to one or more current images taken by the camera 106 of the imaging device 100. This allows a clinician to identify any changes in an anatomical site of interest captured by the imaging device 100 from a prior exam of the anatomical site of interest.

When the imaging device 100 is attached to an otoscope, the image processing application 408 can analyze the images captured by the imaging device 100 for screening of ear infections such as acute otitis media and otitis media with effusion, perforation of the tympanic membrane, foreign bodies in the ear canal such as wax buildup, and other ear conditions.

When the imaging device 100 is attached to a dermatoscope, the image processing application 408 can analyze the images captured by the imaging device 100 for screening of skin cancer, infectious skin diseases, and other skin conditions.

In some examples, the image processing application 408 selects one image from a plurality of images captured by the camera 106 of the imaging device 100. The image processing application 408 selects the image based on at least one of a quality score, a view of a targeted area of the anatomy, or a detected disease state or condition. For example, the image processing application 408 can include a scoring algorithm that calculates the quality score that can be used to select the image having a highest quality score compared to other images that have lower quality scores. In some examples, the image processing application 408 automatically selects one or more images based on the quality scores. Alternatively, a user of the imaging device 100 can manually select one or more images on the imaging device 100 or on the docking station 400 based on the quality scores of the images calculated by the image processing application 408.

Alternatively, or additionally, one or more images can be manually or automatically selected on the imaging device 100 or on the docking station 400 based on whether the images are detected by the image processing application 408 as having a full view of a targeted area of the anatomy. Images captured by the imaging device 100 that include only partial views of the targeted area of the anatomy can be discarded, while images captured by the imaging device 100 that include full views of the targeted area of the anatomy can be stored locally on the docking station 400 and/or can be transferred to an external database such as a cloud-based storage, or in the EMR 302 of the patient maintained by the EMR system 300.

Alternatively, or additionally, one or more images can be manually or automatically selected on the imaging device 100 or on the docking station 400 based on whether a disease state or condition is detected by the image processing application 408. For example, images that do not include a detected disease state or condition can be discarded, while images that include a detected disease state or condition can be stored locally on the docking station 400 and/or can be transferred to an external database such as the cloud-based storage for storage, or to the EMR 302 of the patient maintained by the EMR system 300.

In some further examples, the image processing application 408 performed on the docking station 400 can include de-identifying the images and video streams received from the imaging device 100. For example, the image processing application 408 can include one or more post-processing operations to remove any data that could identify the patient. Once de-identified, the images and videos received from the imaging device can be used for algorithm development.

In further examples, the image processing application 408 performed on the docking station 400 can include providing video conferencing or remote view such as to consult with a remote specialist who is not physically present in the area where the patient is being examined.

In some further examples, the image processing application 408 performed on the docking station 400 can include image stitching such as stitching together one or more separate images captured by the imaging device 100 to provide a panoramic image having a wider field of view. For example, a panoramic image can include a field of view equal to or greater than 160°.

The method 900 includes an operation 914 of storing the one or more images and/or video streams captured by the camera 106 of the imaging device 100. Operation 914 can also include storing the annotations drawn, typed, or entered on the user interface 418 displayed on the display screen 416. Operation 914 can also include storing one or more analyses by the image processing application 408 of the one or images or video streams.

In some examples, operation 914 includes storing the images, video streams, annotations, and/or analyses locally on the memory device 406 of the docking station 400. Additionally, or alternatively, operation 914 can include storing the images, video streams, annotations, and/or analyses in an external database such as cloud-based storage, or in the EMR 302 of the patient maintained by the EMR system 300 via secure communications over the network 30. In such instances, the images, video streams, annotations, and/or analyses are accessible for future reference such as when the patient returns for a follow-up visit.

The various embodiments described above are provided by way of illustration only and should not be construed to be limiting in any way. Various modifications can be made to the embodiments described above without departing from the true spirit and scope of the disclosure.

What is claimed is:

1. A system for image capture and analysis of an anatomy, the system comprising:
an imaging device including:
a bracket that removably attaches the imaging device to an optical viewing device;
a camera that captures images through an eyepiece of the optical viewing device;
a first display screen that displays the images captured by the camera;
a first transceiver that transfers the images captured by the camera to another device; and
a power source that powers the camera, the first display screen, and the first receiver; and
a docking station including:
docking port that supports the imaging device;
a charger positioned inside the docking port for charging the power source of the imaging device;
a second transceiver that receives the images captured by the camera of the imaging device;
a second display screen that displays the images captured by the camera of the imaging device, the second display screen of the docking station being larger than the first display screen of the imaging device; and
a controller including a processing circuitry having a memory storing instructions which, when executed by the processing circuitry, cause the processing circuitry to:
receive the images captured by the camera of the imaging device; and
display the images on the second display screen.

2. The system of claim 1, wherein the memory stores instructions which, when executed by the processing circuitry of the docking station, cause the processing circuitry of the docking station to:
store the images captured by the imaging device in at least one of a local memory device on the docking station and a cloud-based storage.

3. The system of claim 1, wherein the memory stores instructions which, when executed by the processing circuitry of the docking station, cause the processing circuitry of the docking station to:
transfer the images captured by the camera of the imaging device to an external database.

4. The system of claim 1, wherein the memory stores instructions which, when executed by the processing circuitry of the docking station, cause the processing circuitry of the docking station to:
receive annotations on the images displayed on the second display screen; and
store the annotations in at least one of a local memory device on the docking station and a cloud-based storage.

5. The system of claim 1, wherein the memory stores instructions which, when executed by the processing circuitry of the docking station, cause the processing circuitry of the docking station to:
analyze the images captured by the camera of the imaging device.

6. The system of claim 1, wherein the analysis of the images captured by the camera of the imaging device includes screening for a disease state or condition of the anatomy.

7. The system of claim 6, wherein the analysis of the images captured by the camera of the imaging device includes using artificial intelligence.

8. The system of claim 1, wherein the memory stores further instructions which, when executed by the processing circuitry of the docking station, cause the processing circuitry of the docking station to:
receive data identifying the patient;
communicate the data identifying the patient to the imaging device.

9. A method of image capture and analysis of an anatomy, the method comprising:
communicating data identifying a patient to an imaging device, the imaging device removably attached to an optical viewing device, the optical viewing device includes at least one of an ophthalmoscope, an otoscope, and a dermatoscope;
receiving images of the anatomy from the imaging device, the images being captured by a camera of the imaging device that captures the images viewed through an eyepiece of the optical viewing device;
displaying the images of the anatomy on a display screen on a docking station; and
analyzing the images of the anatomy.

10. The method of claim 9, further comprising:
displaying the images of the anatomy on a display screen on the imaging device, the display screen on the imaging device being smaller than the display screen on the docking station.

11. The method of claim 9, further comprising:
storing the images captured by the imaging device in at least one of a local memory device on the docking station and a cloud-based storage.

12. The method of claim 9, further comprising:
transferring the images captured by the imaging device to an external database.

13. The method of claim 9, further comprising:
receiving annotations on the second display screen; and
storing the annotations in at least one of a local memory device on the docking station and a cloud-based storage.

14. The method of claim 9, wherein analyzing the images includes selecting one image from a plurality of images captured by the camera of the imaging device, the one image being selected based on at least one of a quality score, a view of a targeted area of the anatomy, or a detected disease state or condition.

15. The method of claim 9, wherein analyzing the images includes screening for a disease state or condition of the anatomy, and the analysis includes using artificial intelligence.

16. A docking station for an imaging device that is configured to capture images through an eyepiece of an optical viewing device, the docking station comprising:
docking port configured to support the imaging device;
a charging unit positioned inside the docking port for charging a power source of the imaging device;
a transceiver for receiving images captured by a camera of the imaging device;
a display screen configured to display the images received from the imaging device; and
a controller including a processing circuitry having a memory storing instructions which, when executed by the processing circuitry, cause the processing circuitry to:
receive data identifying the patient;
communicate the data identifying the patient to the imaging device;
receive the images captured by the camera of the imaging device, the images being received as raw, unprocessed data;
display the images on the display screen; and
analyze the images captured by the camera of the imaging device.

17. The docking station of claim 16, wherein the memory stores further instructions which, when executed by the processing circuitry, cause the processing circuitry to:
store the images captured by the camera of the imaging device locally on the docking station.

18. The docking station of claim 16, wherein the memory stores further instructions which, when executed by the processing circuitry, cause the processing circuitry to:
transfer the images captured by the camera of the imaging device to an external database.

19. The docking station of claim 16, wherein the memory stores instructions which, when executed by the processing circuitry, cause the processing circuitry to:
receive annotations on the images displayed on the second display screen; and
store the annotations in a local memory on the docking station or in an external database.

20. The docking station of claim 16, wherein the analysis of the images captured by the camera of the imaging device includes selecting one image from a plurality of images captured by the camera of the imaging device, the one image being selected based on at least one of a quality score, a view of a targeted area of the anatomy, or a detected disease state or condition.
